(19) United States
(12) Patent Application Publication
(10) Pub. No.: US 2025/0226105 A1
(43) Pub. Date: Jul. 10, 2025

(54) Disease Progression Prediction

(71) Applicant: Sanofi, PARIS (FR)

(72) Inventors: Hozefa A. DIVAN, Reading, MA (US); Sachin Mathur, Boxborough, MA (US); Cliona Marie Molony, Boston, MA (US)

(21) Appl. No.: 18/850,775

(22) PCT Filed: Mar. 22, 2023
(86) PCT No.: PCT/EP2023/057286

§ 371 Date: Sep. 25, 2024

(30) Foreign Application Priority Data
Mar. 25, 2022 (EP) 22315075.6

(51) Int. Cl. G16H 50/30; G16H 10/60; G16H 20/10; G16H 20/40; G16H 40/67; G16H 50/50; G16H 50/70

(52) U.S. Cl. CPC G16H 50/30; G16H 10/60; G16H 20/10; G16H 20/40; G16H 40/67; G16H 50/50; G16H 50/70

(57) ABSTRACT

Implementations are presented for predicting likelihood of a patient progressing to an advanced stage of a particular medical condition, e.g., a known medical disease. The implementations provide a predictive model and a cluster analysis method. The predictive model and the cluster analysis method can be performed in parallel, or in sequence. Alternatively, only one of the predictive model and the cluster analysis method may be performed, for example, to increase the processing speed and reduce the use of hardware resources.

### CROSS-REFERENCE TO RELATED APPLICATIONS

The present application is the national stage entry of International Patent Application No. PCT/EP2023/057286, filed on Mar. 22, 2023, and claims priority to Application No. EP 22315075.6, filed on Mar. 25, 2022, the disclosures of which are incorporated herein by reference.

### BACKGROUND

The likelihood of progressing to an advanced stage of a disease can differ for different patients. While it may take a few years for a first patient to progress from a stage one cancer to a stage four cancer, it may take only a few months for a second patient to experience such progress, while a third patient may never see an advancement of the cancer beyond stage two. Depending on how likely and how far a disease may advance in a particular patient, different medical procedures or treatments may be prescribed for the patient. In any case, an early diagnosis of the disease can often improve the chance of recovery, and reduce the fatal risks resulted from an advancement of the disease.

### SUMMARY

Implementations of the present disclosure include computer-implemented methods and systems for analyzing and predicting progress of a medical condition, e.g., a particular disease, in patients. The analysis and the prediction are based on clinical characteristics of the patients measured or observed at multiple points in time during the progression of the disease. Accordingly, the present implementations can predict a patient's likelihood of progressing to an advanced stage of a disease based on the patient's current or past clinical characteristics (also referred to as “medical features” herein). Clinical characteristics of a patient can include one or more of information on demographics (e.g., age, gender, place of birth, and region of living), medical history (e.g., past or present diagnoses, medical prescriptions, and medical procedures), biomarker information (e.g., tumor size), body mass index, and life style of the patient (e.g., smoking and drinking habits).

In some of the present implementations, the predictive model predicts the likelihood of a patient to progress to an advanced stage of a disease based on clinical characteristic of the patient measured at one or more points in time. The model is trained by using training data that includes clinical characteristics of a set of training patients. The clinical characteristics of each patient is measured at multiple points in time during the patient's disease progression journey. Once the model is trained, the model can predict the likelihood of the disease progression, for example, to a predetermined advanced stage, for a patient, e.g., a test patient.

In some implementations, a cluster analysis is performed to find patterns in medical features, and thus, to identify medical features that correlate with the progression of the disease. The results of cluster analysis can be used to find the clinical features associated with disease progression and proportion of patients associated with such features. The clinical features can be used in the predictive model to boost up the accuracy in the predictions.

The present disclosure also provides one or more non-transitory computer-readable storage medium coupled to one or more processors and having instructions stored thereon which, when executed by the one or more processors, cause the one or more processors to perform operations in accordance with implementations of the methods provided herein.

The present disclosure further provides a system for implementing the methods provided herein. The system includes one or more processors, and a computer-readable storage medium coupled to the one or more processors having instructions stored thereon which, when executed by the one or more processors, cause the one or more processors to perform operations in accordance with implementations of the methods provided herein.

Methods in accordance with the present disclosure may include any combination of the aspects and features described herein. That is, methods in accordance with the present disclosure are not limited to the combinations of aspects and features specifically described herein, but also include any combination of the aspects and features provided.

Among other advantages, the present implementations provide the following benefits. Since the present implementations train the predictive model based on the progression journeys of the patients, the present implementations provide a more accurate estimate of a progression of a disease for a particular patient compared to conventional methods that use single or isolated treatments and evaluation outcomes such as survival and recurrence rates. Further, the prediction that the present systems provide can include time, stage of advancement, or any other parameter in predicting progression of the disease. For example, the system can indicate when and with how much accuracy a particular patient will progress from one stage of cancer to another (e.g., from a stage one cancer to a stage two cancer, from a stage one cancer to a stage three cancer, from a stage one cancer to a stage four cancer, from a stage two cancer to a stage three cancer, from a stage two cancer to a stage four cancer, etc.).

In addition, the present implementations use clinical characteristics of patients in making the predictions. Compared to conventional methods that consider only treatment features, such as survival and recurrent rates, the present implementations provide a personalized prediction for patients. The personalized predictions provide an estimate of a particular patient's likelihood or journey in progressing to an advanced stage of a disease, based on the medical characteristics specific to the particular patient. The advanced stage of the disease can be predefined; it can be a specific stage of the disease, which can be defined based on how much the disease has spread in the patient's body, based on the size of a tumor in the patient's body, based on medical treatments that the patient has as options to take to treat the disease, based on the medical treatments or medications that the patient has undertaken, etc. The implementation can predict different stages of the disease's progression, based on each stage's respective predefined parameters.

The implementations can use a predictive model to estimate the likelihood of the disease progression. The implementations can also use a cluster-based analysis to complement the predictive model building by confirming or rejecting feature that the predictive model has identified. One can confirm if findings overlap between the two approaches, detect batch effects and check if there is any complementary value addition. Thus, the two models can be used to confirm, reject, or modify the outcome of each other.

The details of one or more implementations of the present disclosure are set forth in the accompanying drawings and the description below. Other features and advantages of the present disclosure will be apparent from the description and drawings, and from the claims.

### DESCRIPTION OF THE FIGURES

FIG. 1 depicts an example system that can be used to execute implementations of the present disclosure.

FIGS. 2A and 2B are example processes that can be executed by a system in accordance with implementations of the present disclosure.

FIG. 3 is an example process that can be executed by a clustering module in accordance with implementations of the present disclosure.

FIG. 4 shows a schematic diagram of an example computing device and a mobile computing device that can perform the methods described in the present disclosure.

Like reference symbols in the various drawings indicate like elements.

### DETAILED DESCRIPTION

The present disclosure provides methods and systems for predicting likelihood of progression of a medical condition, e.g., a disease, in a patient to one or more advanced stages. The disease can be any known disease, for example, any type of cancer or tumor. In an example, the disease is Cutaneous Squamous Cell Carcinoma (CSCC), which is the second most common type of skin cancer that currently causes about 7000 deaths per year in United States due to the advanced stage of the cancer.

The present implementations can reduce diagnosis delays, and thus, lower the chance of a patient progressing to an advanced stage of the disease, and increase the treatment and survival rate of the patient. For example, the common diagnosis and treatment procedure for CSCC is that a dermatologist initially diagnoses the patient with the cancer, and depending on the dermatologist's diagnosis, the patient is referred to an oncologist. This process is both time consuming, and subjective—i.e., subject to the dermatologist's observations. The present implementations reduce (or even eliminate) the effect of both of these issues. The procedure disclosed herein can be used as early as in the patient's initial diagnosis of the disease, and does not suffer from a particular doctor's misdiagnosis or delayed diagnosis.

In some implementations, the cluster analysis is used to find patterns in the data and give specifics of the features that are important in those patterns. It can be an unsupervised machine learning approach where the machine does not know if the patient has an advanced form of the disease. Any feature obtained from the cluster analysis can be a predominant pattern in the data.

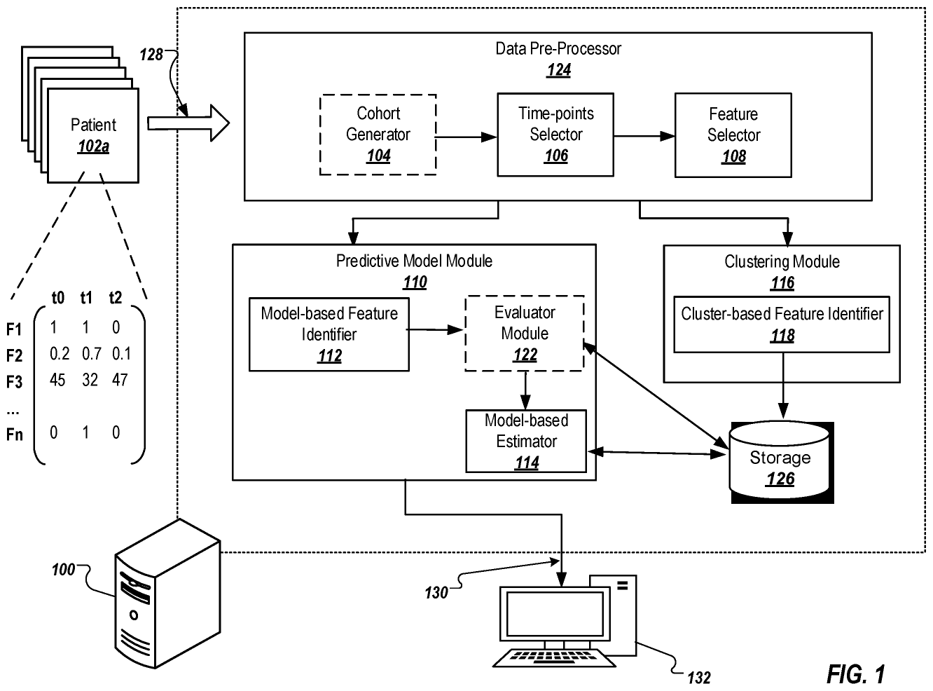

The predictive model, on the other hand, is capable of specifically differentiating between an advanced form of a disease versus a non-advanced form of the disease. In some implementations, the features identified by the predictive model is compared to the features identified by the cluster analysis. If the features identified in the predictive model overlap with the features identified through the cluster analysis, then the model can be recognized as capturing the predominant patterns in the data. If there are mismatches between the features, the predictive model may be modified to reduce (e.g., minimize or eliminate) the mismatch. FIG. 1 depicts an example system 100 that can be used to execute implementations of the present disclosure. System 100 receives input data 128 and provides output 130. During a training phase, input data 128 is the clinical characteristics (i.e., medical features) of a set of training patients 102a-102n, and output data 130 can be a set of identified predictive features or cluster of features. During an interference phase, input data can be the medical characteristics (i.e., features) of a particular patient, and output 130 is a prediction of the particular patient's advancement in a disease, e.g., an estimate of the likelihood of the patient progressing to a predetermined advanced stage of the disease, or a prediction of a journey for the patient to progress to the advanced stage of the disease.

System 100 includes data pre-processor module 124, predictive model module 110, and clustering module 116. System 100 can also include storage 126 and evaluator module 122. System 100 is capable of communicating with external devices, e.g., external device 132, to send or receive data, for example, to receive the input data, to store the output data, and/or to transmit the output for presentation to a user.

Data pre-processor 124 performs initial operations on the input data, for example, to clean up, to categorize, to identify particular features of interest, etc. Data pre-processor 124 can have one or more sub-modules, e.g., cohort generator 104, time-points mapper 106, feature identifier 108, etc. Some of these sub-modules may operate only at particular phases of system 100's operation. For example, cohort generator 104 may operate only at the training phase. Data pre-processor 124 transmits the pre-processed data to one or both of predictive model module 110 and clustering module 116.

When system 100 is in a training phase, each of predictive model module 110 and clustering module 116 can use the input data to identify respective predictive features that later can be used in predicting a particular patient's likelihood of progressing to an advanced stage of a disease. The identified predictive features can be stored in a data storage, which can be part of system 100, e.g., storage 126, or an external device in communication with system 100, e.g., external device 132. When the system is in an interference phase, each of predictive model module 110 and clustering module 116 predicts a respective likelihood of a particular patient's progression to an advanced stage of a disease based on information of the respective features that predictive model module 110 or clustering module 116 identified in the training phase.

In the training phase, system 100 receives training data as medical features (e.g., F1, F2, . . . , Fn) of multiple patients, e.g., patients 102a-102n. The medical features of each patient include information indicating whether the patient progressed to an advanced stage of a particular disease.

The data pre-processor 124 divides the training data into multiple cohorts of patients. For example, cohort generator 104 divides patients 102a through 102n to an advanced cohort and a non-advanced cohort. The advanced cohort includes patients whose disease has progressed to a predetermined advanced stage. The non-advanced cohort includes patients whose disease has not progressed to the predetermined advanced stage. Each medical feature (F1, F2, . . . , Fn) of a patient is a clinical characteristic of the patient that is measured at multiple points in time during the disease progression journey of the patient. In the example training data shown in FIG. 1, each of the medical features F1 through Fn for patient 102a is measured at three points in time t0, t1, and t2, during the disease progression journey of patient 102a. But there is no limit on how many points in time to use for measuring, recording, or using the medical features.

Since each patient can have a different disease progression journey, the points in time when the measurements happen can also differ from a first patient to a second patient. To compare relevant medical features with each other, time-points mapper 106 of data pre-processor module 124 identifies the absolute points in time t0, t1, and t2 that are of interest, and converts them to relative time-points T0, T1, T2 for each journey with respect to when the journey starts and when it ends.

For example, while a journey for a first patient may take five years to advance from a stage one cancer to a stage four cancer, the journey for a second patient may take two years to progress from the stage one cancer to the stage four cancer. The number and timing of the feature measurements and diagnoses that the first patient did during the first journey can differ from the number and timing of the feature measurements that the second patient did during the second journey. Thus, time-points mapper 106 converts the absolute measurement times (t0, t1, t2) to relative time-points (T0, T1, T2) so that system 100 can compare features that were measured at relative time-points with respect to each journey.

Each of the relative time-points is a predefined point in time during the disease progression journey of a patient. The disease progression journey for a patient can start from a specified period of time before or after an initial diagnosis of the disease for the patient. For example, T0 can indicate an initial point of the journey, which can be a year before the initial diagnosis. The journey can end at a predefined ending time-point. For patients 102a-102n that progressed to a specific advanced stage of the disease, the ending time-point can be set as when the patient is diagnosed with the advanced stage. For patients that have not progressed to the advanced stage yet, the ending time-point can be defined with respect to, e.g., as a median of, the ending time-points of the patients who did progress to the advanced stage. In the examples presented above, T2 can represent the respective ending time-point of the journey for each training patient. T1 can be a predetermined value between T0 and T2, for example, six months post the initial diagnosis.

Alternatively, or in addition, the mapping from the absolute points in time to the relative time-points may have been done before the training data enters system 100. For example, each of points in time t0, t1, and t2 for patient 102a may indeed be a relative time-point with respect to the patient 102a's disease progression journey.

Once the relative time-points are obtained, time-points mapper 102 selects a predetermined number (e.g., three) or a particular set of the time-points so that the system would focus on the features measured on those time-points. For example, the particular set can be T0 as the starting point of a journey, T1 as six months into an initial diagnosis of the disease, and T2 as an end-point of the journey. Thus, if medical features of patient 102j (which is a patient among training data patients 102a-102n) have been measured at a plurality of points in time that are mapped to time-points T0, T1, T2, and T3, the system would skip the features that are measured at T3. Depending on the desired processing speed and accuracy, time-points mapper 106 may select more or fewer number of time-points. Selecting fewer number of time-points would increase the processing speed, while selecting more numbers would improve the accuracy in identifying the proper predictive features and in estimating the disease advancement likelihoods. Alternatively, or in addition, an operator can select or can be involved in selecting one or more of the time-points.

Time-points mapper 106 can perform the mapping and the selecting in any order. It can first map and then select, first select and then map, or map and select in parallel. For example, time-points mapper 106 can target and select only particular points in time (e.g., the start of the journey, six months from the first diagnosis, and the end of the journey), and map only those particular points in time to respective time-points.

Data pre-processor 124 can also select one or more features from among multiple features that are included in the training data. For example, feature selector 108 may select features F1 through Fj from among features F1 through Fn measured for patient 102a. Data pre-processor 124 can select the same features (e.g., features F1 through Fj) at the same time-points (e.g., time-points T0, T1, T2) for all training patients 102a-102n.

Data pre-processor 124 provides the generated cohorts, e.g., the advanced and the non-advanced cohorts, the time-points, e.g., time-points T0, T1, T2, and the selected features, e.g., features F1 through Fj, to each of predictive model module 110 and clustering module 116. Continuing in the training phase, predictive model module 110 trains a predictive model by using the pre-processed training data. Training of the predictive model includes identifying predictive features. Predictive features can be features that differentiate between the patients in the advanced cohort and the non-advanced cohort at various time-points.

To determine whether a particular feature in the selected features F1-Fj is a predictive features, model-based (MB) feature identifier 112 can determine a first number of patients in the non-advanced cohort that have the particular feature, and a second number of patients in the advanced cohort that have the particular feature, at each of the selected time-points T0, T1, and T2.

MB feature identifier 112 obtains a delta value for each time-point by calculating a difference between the first number and the second number at that time-point. MB feature identifier 112 determines if the feature is identified at particular times, e.g., T0 and T1, or when moving from a first time-point, e.g., T0, to a second time-point, e.g., T2, later than the first time-point. In some implementations, MB feature identifier 112 uses an iterative partitioning procedure to partition patients, e.g., two sets, based on the features that best distinguish patients who progressed to an advanced form of a disease from patients who did not progress to the advanced form. The partitioning can be based on respective feature values of one or more features. For example, MB feature identifier 112 can use the features of using anti-proliferative, and not taking medications to partition the patients. This is repeated until either distinct sets of advanced and non-advanced patients are formed, or until the patients can no longer be distinguished based on the feature values of the particular feature. MB feature identifier 112 can repeatedly apply this procedure to all features to obtain various combinations of features to distinguish advanced and non-advanced patients. The MB feature identifier 112 can also rank the features in terms of their ability to distinguish between the advanced and non-advanced patients.

In some implementations, module 112 checks if a particular feature is related to the targeted disease in presence of other features and makes a statistical determination on the importance of the particular feature. The importance is determined based on how often the particular feature occurs among patients suffering from the targeted disease, or based on a value or a range of the values of the particular feature among the patients that suffer from the particular disease.

In some implementations, the delta value gives additional information on the particular feature's relative importance between multiple time points (e.g., T0 through T2). In some implementations, the level of importance of the particular feature with respect to the targeted disease can be confirmed if both the predictive model and the changes in the delta value among multiple time-points confirm that the particular feature is an indicator of progression in the targeted disease.

As a first example, if 1000 patients from the advanced cohort and 500 patients from the non-advanced cohort were using medication X at T0, the delta value for T0 for the feature of “using medication X” would be 500. If 1500 patients from the advanced cohort and 800 patients from the non-advanced cohort were using the medication X at T2, then the delta value at T2 for this feature would be 700. Assuming T2 is later than T0, the delta value for this feature increased for 40% from T0 to T2 (i.e., (700-500)/500). Now if the threshold value is 20%, then MB feature identifier 112 identifies the feature of “using medication X” as a predictive feature because 40% increase is more than the 20% threshold. But, as a second example, if only 950 patients from the non-advanced cohort were using the medication X at T2, then “using medication X” would not be identified as a predictive feature because the delta value increased for only 10% (i.e., (550-500)/500) from T0 to T2.

In some implementations, MB feature identifier 112 determines one or more respective predictive powers for each predictive feature. In some embodiments, the predictive power of a feature can be calculated as a difference of the feature's value measured at different time-points, e.g., T2 and T0, or T1 and T0. In some embodiments, the predictive power can be calculated as the difference normalized by a reference value, e.g., the value of the feature measured at T0. For example, in the first example discussed in the preceding paragraph, the predictive power of the feature of “using medication X” would be 40% for predicting medical features at T2 based on features measured at T0. Predictive model module 110 can later use the predictive power of each feature in the interference phase, when using the features to predict the disease progression in a new patient.

In some implementations, MB feature identifier 112 identifies the predictive features by using a tree-based approach on respective values of medical features received from data pre-processor 124, to differentiate patients in the advanced cohort from patients in the non-advanced cohort at various time-points, for example, according to the procedure explained earlier. MB feature identifier 112 makes the differentiation in an iterative manner based on the respective values of the medical features.

Predictive model module 110 can store information of the predictive features in a data storage, e.g., storage 126, or transmit the predictive features for presentation on another device, e.g., external device 132. The information of a predictive feature can include respective identifier, predictive power, measured time-points, etc., associated with the predictive feature. In the interference phase, system 100 receives medical features of a particular patient, e.g., a new patient, and predicts the likelihood of the disease progressing to an advance stage in that patient. Data pre-processor 124 can optionally pre-process the medical features of the particular patient, for example, to select the particular time-points and features that predictive model 100 used to identify the predictive features. In some implementations, the pre-processed data includes only the predictive features whose information data pre-processor 124 can obtain from storage 126 or from predictive model module 110. In some implementations, the pre-processed data includes only a subset of the time-points that predictive model 100 used to identify predictive features.

In some implementations, system 100 does not use one or more sub-modules of data pre-processor 124 in the interference phase. For example, rather than using feature selector 108, predictive model module 110 works from the medical features of the particular patient that system 100 received and skips any medical feature that does not associate with any of the predictive features.

In the interference phase, model-based (MB) estimator 114 of predictive model module 110 can use the predictive model that was trained in the training phase to predict a model-based likelihood of the disease progression for the particular patient. The model-based likelihood can be predicted as an estimate of the particular patient's chance in progressing to a predetermined advanced stage of the disease.

The input data to MB estimator 114 is the pre-processed data from data pre-processor 124, and the predictive features identified by the MB feature identifier 112. The output data of MB estimator 114 is the model-based likelihood. MB estimator 114 generates the output by retrieving, for example, from storage 126, the information of the predictive features that MB feature identifier 112 had identified in the training phase. MB estimator 114 then uses the predictive features on the trained predictive model and applies the model to the medical features of the particular patient to estimate the model-based likelihood.

In some implementations, MB estimator 114 uses each of the predictive features according to the predictive power associated with the predictive feature. For example, if MB feature identifier 112 had determined a 20% predictive power for a first predictive feature, and a 40% predictive power for a second predictive feature with respect to a particular disease, MB estimator 114 considers the second predictive feature as a more determinative factor than the first predictive feature in estimating the likelihood of progressing to an advanced stage of the disease. MB estimator 114 can provide the estimated likelihood to an external device, for example, for presentation or storage.

. Clustering module 116 includes a Cluster-based (CB) feature identifier 118 that clusters patients 102a-102n based on a first subset of features, e.g., features F2, F4, . . . , F10 measured at a particular time point, e.g., T0 or T2. In contrast to the training phase in the predictive model, clustering module 116 does not use information on the progression of the disease for a patient. For example, the occurrence of Advanced cSCC or non-Advanced cSCC can be hidden from the clustering algorithm. The resulting clusters represent a respective subset of patients that share predetermined similarities in the first subset of features. In some examples, a feature is measured on a binary basis, and the similarity means a presence or an absence of the feature on patient; for example, presence or absence of a medication in patients. In some examples, a feature is measured in a continuous basis, and a similarity means how closely the feature is presented in two different patients. For example. As another example, age of patients at T0 time point, e.g., one feature indicates 30-40 years old patients at T0, another features indicates 40-45 years old patients at T0.

From among the generated clusters, CB feature identifier 118 identifies one or more subset of clusters that each has a greater than a threshold number of patients (for example, 50 patients, or 60% of the patients in the cluster) whose disease progressed to the predetermined advanced stage. For each cluster in the identified subsets of clusters, CB feature identifier 118 identifies a respective second subset of features (also referred to as a subset of “dominant” features) that are common between patients in the cluster. In some implementations, a feature can be in a subset of dominant features if more than a second threshold number, e.g., more than 70%, of the patients in a cluster have that feature. In some implementations, a feature can be in a subset of dominant features if more than the second threshold number of patients that had the advanced stage of the disease in the cluster have the feature.

CB feature identifier 118 outputs the second subsets of features (i.e., subsets of dominant features). The output can be transmitted to evaluator module 122, be stored in a storage device 126, or be transmitted to an external device (e.g., 132) for presentation or further processing. In some implementations, clustering module 116 uses the output of CB feature identifier 118 to determine importance of each feature in subsets of dominant features with respect to an advanced form of the disease. For example, clustering module 116 can overlay the disease-related information (e.g., the advancement of the disease) of each patient in the clusters, calculate delta values for each feature within each cluster, and determine the relative importance of a feature or a correlation between the feature and progressing into the advanced form of the disease based on the change of the delta between multiple time points, e.g., T0-T2. Indeed, clustering module 116 determines correlations between features that were observed or measured in patients that had the advanced stage of the disease. In some implementations, the features that are identified as the most important features are picked by the predictive model module 110, e.g., by evaluator module 122, to verify or confirm the predictive features identified by the MB feature identifier 112.

In some implementations, evaluator module 122 compares the dominant features outputted from CM features identifier 118 to the predictive features outputted from MB feature identifier 112, to identify feature mismatches between the dominant features and the predictive features. In case of finding mismatches, e.g., finding that a predictive feature is not included in the dominant features or that a dominant feature is not included in the predictive features, the evaluator module can make changes to reduce or minimize the mismatch.

In some implementations, the changes include editing the predictive features by, e.g., eliminating the extra predictive features that had no match among the dominant features, adding the dominant features that had no match in the predictive features, or making changes to the values corresponding to the predictive features (e.g., age range) to match the values correspond to respective dominant features. Evaluator module 122 can retrieve the predictive features from MB feature identifier 112 or from storage 126. Evaluator module 122 can transmit the modified predictive features to storage 126 to replace the prior predictive features. Evaluator module 122 can transmit the modified or the approved predictive features to MB estimator 114 to be used for likelihood estimation process.

In some implementations, the changes (that evaluator module 122 makes subsequent to finding mismatches between the dominant features and the predictive features) include modifications in the parameters of the predictive model so that the model provides predictive features that also the dominant features identified by 118. In some implementations, the parameters of the predictive model are modified so that the predictive features that the modified model identifies, match the dominant features.

In some implementations, evaluator module 122 evaluates the predictive features based on all dominant features identified by CB feature identifier 118. In some implementations, evaluator module 122 evaluates the predictive features based on dominant features in one or more particular subsets of dominant features identified by the CB feature identifier. For example, a particular subset may include age of the patient at T0, use of a particular medicine, and Mohs surgery. When evaluating the predictive feature of age, evaluator module 122 considers whether the other dominant features (i.e., the use of the particular medicine, and Mohs surgery) included in the subset are also among the predictive features.

In some implementations, evaluator module 122 considers the dominant features included in each subset, as correlated features, and adds the correlation between the corresponding predictive features as well. In the example above, once evaluator module 122 adds Mohs surgery and use of the particular medicine to the predictive features the evaluator module 122 can also assign a correlation between the three features of Mohs surgery, using of the particular medicine, and age, based on the respective subset of dominant features.

CB feature identifier 118 can identify one or more subsets of dominant features from a particular cluster. For example, assuming that the second threshold number is 70%, if more than 70% of patients in a cluster have all three features of male, HIV positive, and actinic keratosis, CB feature identifier 118 can create a subset including these three dominant features. Also, if more than 70% of patients in the same cluster have features of being older than 50 years old, used a particular medication (e.g., topical antiproliferatives immunomodulators), had a particular medical procedure (e.g., incisional biopsy of ear, lip, or eyelid), and are of a particular race, CB feature identifier 118 includes these four features in another subset of dominant features that is formed from the same particular predictive cluster.

Each subset of dominant features can be associated with a particular time-point associated with when the respective features in that subset were measured or observed. For example, a first subset can include features of HIV positive and actinic keratosis measured at a point in time associated with time-point T0, while a second subset can include the same features of HIV positive and actinic keratosis measured at a later point in time associated with time-point T1. CB feature identifier 118 can associate each subset of dominant features to a respective likelihood of the disease progressing to the advanced stage. CB feature identifier 118 associates a likelihood to a predictive subset of features by contrasting how many patients shared the same value (or share values in a specific range) for a feature in the group of patients that progressed to the advanced form of the disease and the group of patients that did not progress to the advanced form of the disease.

CB feature identifier 118 can output information of the subsets of dominant features to be stored, e.g., in storage 126 or in an external data storage, or to be transmitted for presentation to a user, e.g., by external device 132. CB feature identifier 118 can transmit the subset of dominant features to evaluator module 122 that, as explain above, can verify the accuracy of the predictive features identified by the MB feature identifier 112.

In some implementations, each of predictive model module 110 and the clustering module 116 transmits its outputs to an external device 132, for example, for presentation to a user, or for further processes. Predictive model module 110 can transmit the calculated likelihood, or the identified predictive features to the external device. Clustering module 116 can transmit the subsets of dominant features identified from the created clusters to the external device. While the description above provided methods for predicting a likelihood of a patient progressing to a predetermined advanced stage of a disease, similar approaches can be taken to predict a timeline for the progress of the disease for the patient. For example, a patient's disease progression by time-points T1 and T2 can be estimated based on the data measured before points in time t1 and t2 associated with T1 and T2, e.g., at point in time to (associated with time-point T0); and the patient's disease progression by time-point T2 can be estimated based on the data measured before the point in time t2 associated with time-point T2, e.g., at time points t0 and t1 (associated with time-points T0 and T1). The more time-points that time-points mapper 106 selects, the more number of points in time can be used as the reference for the timeline in predicting likelihood of progression of the disease on the user. System 100 can be capable of suggesting a medication or a medical procedure for the patient based on one or more of the predicted likelihoods. For example, based on the advancement of the disease on a particular patient, and the clinical characteristics of the patient, the system can suggest one or more medications or medical procedures that were found more effective or commonly used among the training patients 102a-102n that did not progress to the advanced stage of the disease.

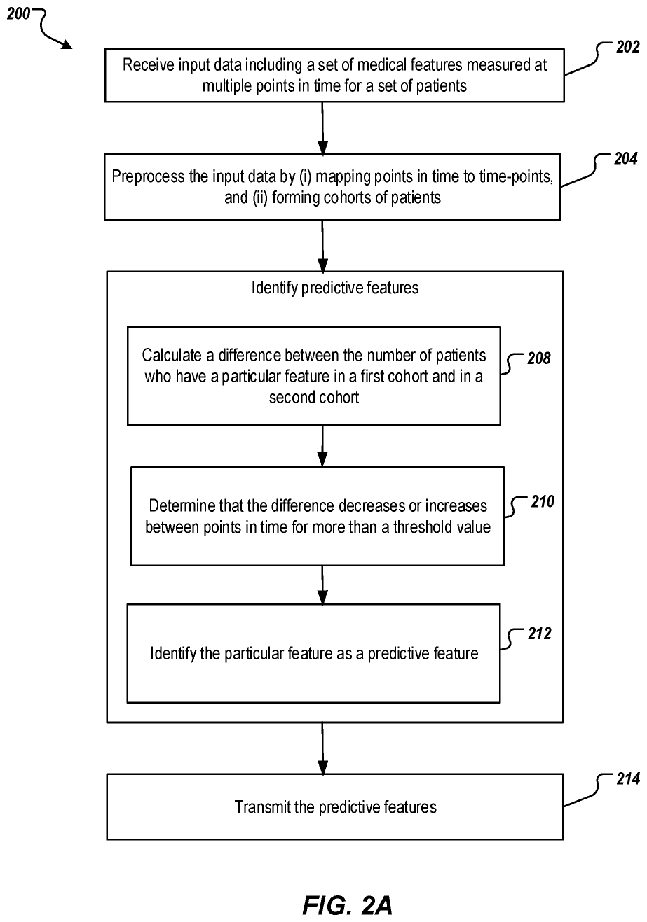

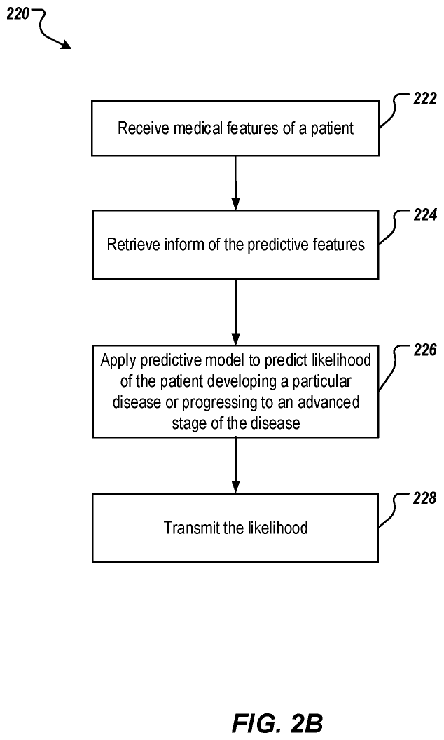

FIGS. 2A and 2B depict example processes 200 and 220 that can be executed by a computing system, e.g., system 100, in accordance with implementations of the present disclosure. Process 200 can be executed to provide a set of predictive features for training a predictive model. Process 220 can be executed to predict a likelihood of a patient having or developing a particular disease, or an advanced stage of a particular disease based on the predictive features.

The system receives input data including a set of medical features associated with a set of patients (202), e.g., as training data. The system can receive the set of medical features though communication with an external device. Each feature in the set of medical features is measured at multiple points in time throughout a disease progression journey of a respective patient in a set of patients (e.g., 102a-102n).

The system pre-processes the input data (204). The pre-processing includes mapping the points in time to respective time-points and forming cohorts of patients. The mapping includes associating each point in time to a respective time-point that represent the relativeness of the point in time to the overall disease progression journey of the patient. The cohorts can include an advanced cohort and a non-advanced cohort. The advanced cohort can include patients whose disease progressed to a predetermined advanced stage. The non-advanced cohort can include patients whose disease has not progressed to the predetermined advanced stage. The system identifies one or more predictive features from the medical features (206), for example, by applying a predictive model on the set of medical features. The predictive features differentiate between the advanced cohort and the non-advanced cohort at various time-points. To determine whether a particular feature in the set of medical features is a predictive feature for a particular disease, the system calculates a difference between the number of patients who have the particular feature in the advanced (or first) cohort and the number of patients who have the particular feature in the non-advanced (or second) cohort (208). The system then determines whether the difference in those numbers increases or decreases when moving from a first time-point to a second time-point. If the difference increases or decreases for more than a threshold value (210), the system identifies the particular feature as a predictive feature (212). The module transmits the predictive features (214), for example, to store the predictive features in a storage device, or to present the predictive features on another device, e.g., device 132 in FIG. 1.

In process 220 depicted in FIG. 2B, the system receives medical features of a patient (222), for example, as test data. The system retrieves information of the predictive features (224). The system then applies the predictive model on the medical features of the patient to predict likelihood of the patient developing or having the particular disease, or progressing to an advanced form of the disease (226). The system transmits the likelihood (228), for example, to be stored or presented on another device.

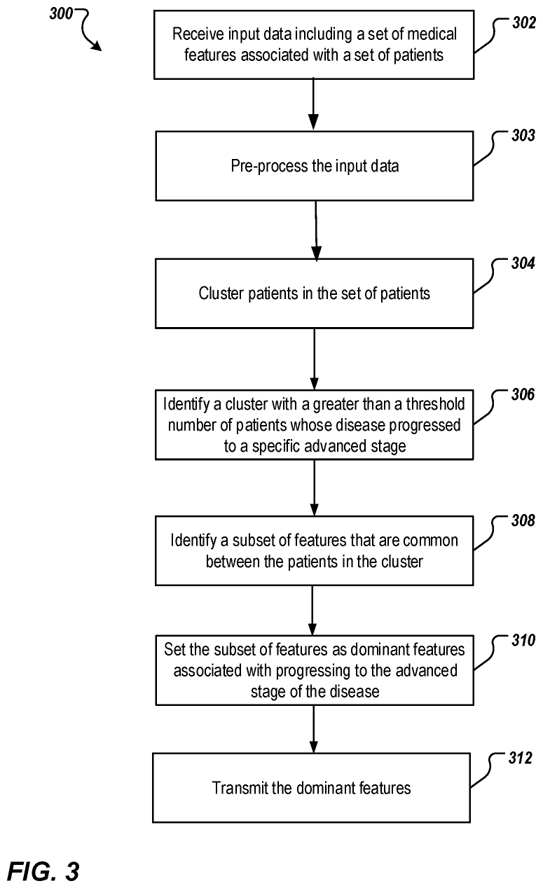

FIG. 3 is an example process that can be executed by a clustering module, e.g., 116, in accordance with implementations of the present disclosure. The module receives a set of medical features (302), for example, as training data. The module can receive the set of medical features though communication with an external device. Each feature in the set of medical features is measured at multiple points (e.g., t0, t1, t2) in time throughout a disease progression journey of a respective patient in a set of patients (e.g., 102a-102n).

The module pre-processes the input data (303). The pre-processing can include mapping the points in time to respective time-points. The mapping includes associating each point in time to a respective time-point (e.g., T0, T1, T2) that represent the relativeness of the point in time to the overall disease progression journey of the patient.

The module clusters the patients in the set of patients (304), for example, based on a first subset of features. The first subset of features can be measured or observed at a particular point in time. Each cluster represents a respective subset of patients that share predetermined similarities in the first subset of features.

The module identifies a cluster with a greater than a first threshold number of patients whose disease progressed to a specific advanced stage (306). The module identifies one or more second subsets of features that are common between patients in the identified cluster (308).

The module sets the identified second subsets of features as dominant features associated with progressing to the advanced stage (i.e., a predetermined advanced form) of the disease. (310). The module can transmit the dominant features (312), for example, for storage or for presentation.

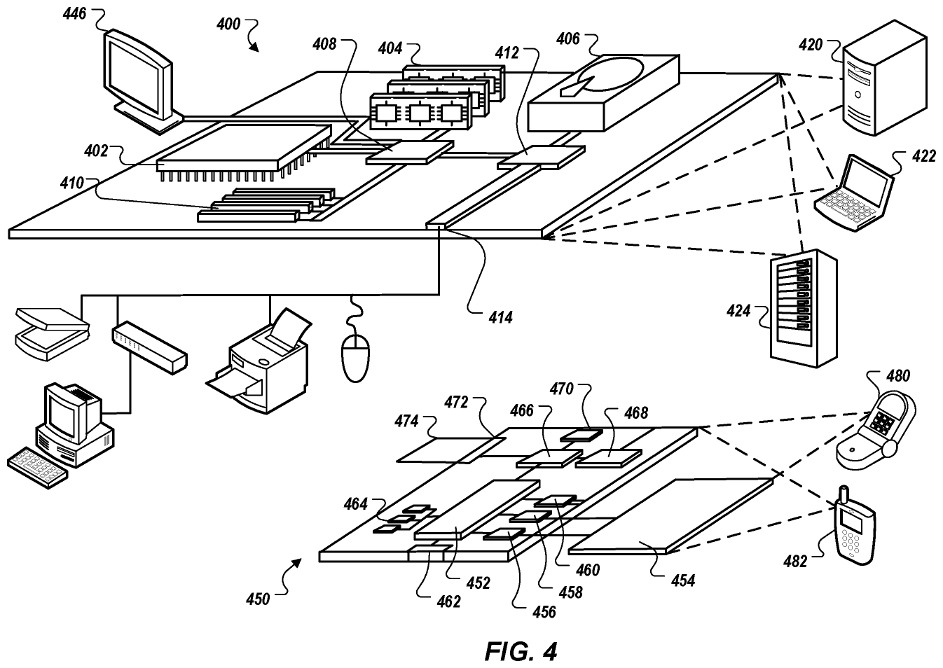

FIG. 4 shows an example of a computing device 400 and an example of a mobile computing device that can be used to implement the techniques described here. For example, system 100 in FIG. 1 can be in the form of the computing device 400, the mobile computing device 450, or a combination of them. The computing device 400 is intended to represent various forms of digital computers, such as laptops, desktops, workstations, personal digital assistants, servers, blade servers, mainframes, and other appropriate computers. The mobile computing device is intended to represent various forms of mobile devices, such as personal digital assistants, cellular telephones, smart-phones, and other similar computing devices. The components shown here, their connections and relationships, and their functions, are meant to be exemplary only, and are not meant to limit implementations of the inventions described and/or claimed in this document.

The computing device 400 includes a processor 402, a memory 404, a storage device 406, a high-speed interface 408 connecting to the memory 404 and multiple high-speed expansion ports 410, and a low-speed interface 412 connecting to a low-speed expansion port 414 and the storage device 406. Each of the processor 402, the memory 404, the storage device 406, the high-speed interface 408, the high-speed expansion ports 410, and the low-speed interface 412, are interconnected using various busses, and can be mounted on a common motherboard or in other manners as appropriate. The processor 402 can process instructions for execution within the computing device 400, including instructions stored in the memory 404 or on the storage device 406 to display graphical information for a GUI on an external input/output device, such as a display 416 coupled to the high-speed interface 408. In other implementations, multiple processors and/or multiple buses can be used, as appropriate, along with multiple memories and types of memory. Also, multiple computing devices can be connected, with each device providing portions of the necessary operations (e.g., as a server bank, a group of blade servers, or a multi-processor system).

The memory 404 stores information within the computing device 400. In some implementations, the memory 404 is a volatile memory unit or units. In some implementations, the memory 404 is a non-volatile memory unit or units. The memory 404 can also be another form of computer-readable medium, such as a magnetic or optical disk.

The storage device 406 is capable of providing mass storage for the computing device 400. In some implementations, the storage device 406 can be or contain a computer-readable medium, such as a floppy disk device, a hard disk device, an optical disk device, or a tape device, a flash memory or other similar solid state memory device, or an array of devices, including devices in a storage area network or other configurations. A computer program product can be tangibly embodied in an information carrier. The computer program product can also contain instructions that, when executed, perform one or more methods, such as those described above. The computer program product can also be tangibly embodied in a computer- or machine-readable medium, such as the memory 404, the storage device 406, or memory on the processor 402.

The high-speed interface 408 manages bandwidth-intensive operations for the computing device 400, while the low-speed interface 412 manages lower bandwidth-intensive operations.

Such allocation of functions is exemplary only. In some implementations, the high-speed interface 408 is coupled to the memory 404, the display 416 (e.g., through a graphics processor or accelerator), and to the high-speed expansion ports 410, which can accept various expansion cards (not shown). In the implementation, the low-speed interface 412 is coupled to the storage device 406 and the low-speed expansion port 414. The low-speed expansion port 414, which can include various communication ports (e.g., USB, Bluetooth, Ethernet, wireless Ethernet) can be coupled to one or more input/output devices, such as a keyboard, a pointing device, a scanner, or a networking device such as a switch or router, e.g., through a network adapter.

The computing device 400 can be implemented in a number of different forms, as shown in the figure. For example, it can be implemented as a standard server 420, or multiple times in a group of such servers. In addition, it can be implemented in a personal computer such as a laptop computer 422. It can also be implemented as part of a rack server system 424. Alternatively, components from the computing device 400 can be combined with other components in a mobile device (not shown), such as a mobile computing device 450. Each of such devices can contain one or more of the computing device 400 and the mobile computing device 450, and an entire system can be made up of multiple computing devices communicating with each other.

The mobile computing device 450 includes a processor 452, a memory 464, an input/output device such as a display 454, a communication interface 466, and a transceiver 468, among other components. The mobile computing device 450 can also be provided with a storage device, such as a micro-drive or other device, to provide additional storage. Each of the processor 452, the memory 464, the display 454, the communication interface 466, and the transceiver 468, are interconnected using various buses, and several of the components can be mounted on a common motherboard or in other manners as appropriate.

The processor 452 can execute instructions within the mobile computing device 450, including instructions stored in the memory 464. The processor 452 can be implemented as a chipset of chips that include separate and multiple analog and digital processors. The processor 452 can provide, for example, for coordination of the other components of the mobile computing device 450, such as control of patient interfaces, applications run by the mobile computing device 450, and wireless communication by the mobile computing device 450.

The processor 452 can communicate with a patient through a control interface 458 and a display interface 456 coupled to the display 454. The display 454 can be, for example, a TFT (Thin-Film-Transistor Liquid Crystal Display) display or an OLED (Organic Light Emitting Diode) display, or other appropriate display technology. The display interface 456 can comprise appropriate circuitry for driving the display 454 to present graphical and other information to a patient. The control interface 458 can receive commands from a patient and convert them for submission to the processor 452. In addition, an external interface 462 can provide communication with the processor 452, so as to enable near area communication of the mobile computing device 450 with other devices. The external interface 462 can provide, for example, for wired communication in some implementations, or for wireless communication in other implementations, and multiple interfaces can also be used.

The memory 464 stores information within the mobile computing device 450. The memory 464 can be implemented as one or more of a computer-readable medium or media, a volatile memory unit or units, or a non-volatile memory unit or units. An expansion memory 474 can also be provided and connected to the mobile computing device 450 through an expansion interface 472, which can include, for example, a SIMM (Single In Line Memory Module) card interface. The expansion memory 474 can provide extra storage space for the mobile computing device 450, or can also store applications or other information for the mobile computing device 450. Specifically, the expansion memory 474 can include instructions to carry out or supplement the processes described above, and can include secure information also. Thus, for example, the expansion memory 474 can be provide as a security module for the mobile computing device 450, and can be programmed with instructions that permit secure use of the mobile computing device 450. In addition, secure applications can be provided via the SIMM cards, along with additional information, such as placing identifying information on the SIMM card in a non-hackable manner.

The memory can include, for example, flash memory and/or NVRAM memory (non-volatile random access memory), as discussed below. In some implementations, a computer program product is tangibly embodied in an information carrier. The computer program product contains instructions that, when executed, perform one or more methods, such as those described above. The computer program product can be a computer- or machine-readable medium, such as the memory 464, the expansion memory 474, or memory on the processor 452. In some implementations, the computer program product can be received in a propagated signal, for example, over the transceiver 468 or the external interface 462.

The mobile computing device 450 can communicate wirelessly through the communication interface 466, which can include digital signal processing circuitry where necessary. The communication interface 466 can provide for communications under various modes or protocols, such as GSM voice calls (Global System for Mobile communications), SMS (Short Message Service), EMS (Enhanced Messaging Service), or MMS messaging (Multimedia Messaging Service), CDMA (code division multiple access), TDMA (time division multiple access), PDC (Personal Digital Cellular), WCDMA (Wideband Code Division Multiple Access), CDMA2000, or GPRS (General Packet Radio Service), among others. Such communication can occur, for example, through the transceiver 468 using a radio-frequency. In addition, short-range communication can occur, such as using a Bluetooth, WiFi, or other such transceiver (not shown). In addition, a GPS (Global Positioning System) receiver module 470 can provide additional navigation-and location-related wireless data to the mobile computing device 450, which can be used as appropriate by applications running on the mobile computing device 450. The mobile computing device 450 can also communicate audibly using an audio codec 460, which can receive spoken information from a patient and convert it to usable digital information. The audio codec 460 can likewise generate audible sound for a patient, such as through a speaker, e.g., in a handset of the mobile computing device 450. Such sound can include sound from voice telephone calls, can include recorded sound (e.g., voice messages, music files, etc.) and can also include sound generated by applications operating on the mobile computing device 450.

The mobile computing device 450 can be implemented in a number of different forms, as shown in the figure. For example, it can be implemented as a cellular telephone 480. It can also be implemented as part of a smart-phone 482, personal digital assistant, or other similar mobile device.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms machine-readable medium and computer-readable medium refer to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term machine-readable signal refers to any signal used to provide machine instructions and/or data to a programmable processor.

To provide for interaction with a patient, the systems and techniques described here can be implemented on a computer having a display device (e.g., a CRT (cathode ray tube) or LCD (liquid crystal display) monitor) for displaying information to the patient and a keyboard and a pointing device (e.g., a mouse or a trackball) by which the patient can provide input to the computer. Other kinds of devices can be used to provide for interaction with a patient as well; for example, feedback provided to the patient can be any form of sensory feedback (e.g., visual feedback, auditory feedback, or tactile feedback); and input from the patient can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical patient interface or a Web browser through which a patient can interact with an implementation of the systems and techniques described here), or any combination of such back end, middleware, or front end components. The components of the system can be interconnected by any form or medium of digital data communication (e.g., a communication network). Examples of communication networks include a local area network (LAN), a wide area network (WAN), and the Internet.

The computing system can include clients and servers. A client and server are generally remote from each other and typically interact through a communication network. The relationship of client and server arises by virtue of computer programs running on the respective computers and having a client-server relationship to each other.

In some embodiments, a method is executed by a system of one or more computers. The method comprises receiving a set of medical features, each feature in the set of medical features is measured at multiple points in time throughout a disease progression journey in a respective patient in a set of patients; mapping each point in time to a respective time-point that represents the relativeness of the point in time to the overall disease progression journey of the patient; forming, from the set of patients, an advanced cohort and a non-advanced cohort, the advanced cohort including patients whose disease progressed to a predetermined advanced form, the non-advanced cohort including patients whose disease did not progress to the predetermined advanced form; applying a predictive model on the set of medical features to identify one or more predictive features that differentiate between the advanced cohort and the non-advanced cohort at various time-points; and storing, in a data storage device, information of the one or more predictive features.

The method can further include receiving, from a client device, data on medical features of a particular patient; retrieving the information of the one or more predictive features from the storage device; applying the predictive model on the data to predict a likelihood of the particular patient progressing to the advanced form of the disease based on the information of the one or more predictive features; and transmitting the likelihood to the client device for presentation. The method can also include applying the predictive model on the data to predict a timeline for a progress of the disease in the particular patient; and transmitting the timeline to the client device for presentation. The method can include suggesting a medication or a medical procedure for the patient based on the likelihood.

The disease progression journey for a patient can start from a predetermined period of time prior to an initial diagnosis of the disease for the patient. The disease progression journey for a first patient in the advanced cohort can end at a point in time when the patient is diagnosed with an advanced form of the disease. The disease progression journey for a second patient in the non-advanced cohort can end at a point in time that is calculated based on median duration of the journeys of the patients in the advanced cohort.

The at least one medical feature can include clinical characteristics of the patients, the clinical characteristics of a patient comprising one or more of age, gender, geographical birth location, medical diagnoses, prescription information, medical procedures, biomarker information, body mass index, smoking and drinking habits, and laboratory test results of the patient. The method can further include clustering patients in the set of patients based on a first subset of features measured at a particular time-point, each cluster representing a respective subset of patients that share predetermined similarities in the first subset of features; identifying a cluster with a greater than a first threshold number of patients whose disease progressed to the predetermined advanced form; identifying a second subset of features that are common between more than a second threshold number of patients in the identified cluster; and associating the second subset of features to the predetermined advanced form of the disease. The method can also include comparing the second subset of features with the one or more predictive features to identify feature mismatches between the second subset of features and the predictive features, and in response: modifying the predictive model to reduce the feature mismatches. The predictive model can be modified so that the one or more predictive features match the second subset of features.

The predictive model can identify the predictive features by using a tree-based approach on respective values of medical features in the set of medical features to differentiate patients in the advanced cohort from patients in the non-advanced cohort at various time-points based on the respective values of the medical features in an iterative manner.

The predictive model can identify a particular feature as a predictive feature by: determining, at each time-point in the multiple time-points, a first number of patients in the non-advanced cohort that have the particular feature, and a second number of patients in the advanced cohort that have the particular feature; calculating, for each time-point, a respective delta value between the first number and the second number at the time-point; and determining that a first delta value calculated for a first time-point is more than a second delta value calculated for a second time-point for more than a specific threshold value, and in response: identifying the particular feature as a predictive feature.

A number of implementations of the present disclosure have been described. Nevertheless, it will be understood that various modifications may be made without departing from the spirit and scope of the present disclosure. Accordingly, other implementations are within the scope of the following claims.

1. A method comprising:
receiving, by a system of one or more computers, a set of medical features, each feature in the set of medical features is measured at multiple points in time throughout a disease progression journey in a respective patient in a set of patients;
mapping each point in time to a respective time-point that represents the relativeness of the point in time to the overall disease progression journey of the patient;
forming, from the set of patients, an advanced cohort and a non-advanced cohort, the advanced cohort including patients whose disease progressed to a predetermined advanced form, the non-advanced cohort including patients whose disease did not progress to the predetermined advanced form;
applying a predictive model on the set of medical features to identify one or more predictive features that differentiate between the advanced cohort and the non-advanced cohort at various time-points; and
storing, in a data storage device, information of the one or more predictive features.

2. The method of claim 1, further comprising:
receiving, from a client device, data on medical features of a particular patient;
retrieving the information of the one or more predictive features from the storage device;
applying the predictive model on the data to predict a likelihood of the particular patient progressing to the advanced form of the disease based on the information of the one or more predictive features; and
transmitting the likelihood to the client device for presentation.

3. The method of claim 2, further comprising:
applying the predictive model on the data to predict a timeline for a progress of the disease in the particular patient; and
transmitting the timeline to the client device for presentation.

4. The method of claim 1, further comprising suggesting a medication or a medical procedure for the patient based on the likelihood.

5. The method of any of claim 1, wherein the disease progression journey for a patient starts from a predetermined period of time prior to an initial diagnosis of the disease for the patient.

6. The method of any of claim 1, wherein the disease progression journey for a first patient in the advanced cohort ends at a point in time when the patient is diagnosed with an advanced form of the disease, and
wherein the disease progression journey for a second patient in the non-advanced cohort ends at a point in time that is calculated based on median duration of the journeys of the patients in the advanced cohort.

7. The method of any of claim 1, wherein the at least one medical feature includes clinical characteristics of the patients, the clinical characteristics of a patient comprising one or more of age, gender, geographical birth location, medical diagnoses, prescription information, medical procedures, biomarker information, body mass index, smoking and drinking habits, and laboratory test results of the patient.

8. The method of any of claim 1, further comprising:
clustering patients in the set of patients based on a first subset of features measured at a particular time-point, each cluster representing a respective subset of patients that share predetermined similarities in the first subset of features;
identifying a cluster with a greater than a first threshold number of patients whose disease progressed to the predetermined advanced form;
identifying a second subset of features that are common between more than a second threshold number of patients in the identified cluster; and
associating the second subset of features to the predetermined advanced form of the disease.

9. The method of claim 8, further comprising comparing the second subset of features with the one or more predictive features to identify feature mismatches between the second subset of features and the predictive features, and in response,
modifying the predictive model to reduce the feature mismatches.

10. The method of claim 9, wherein the predictive model is modified so that the one or more predictive features match the second subset of features.

11. The method of any of claim 1, wherein the predictive model identifies the predictive features by using a tree-based approach on respective values of medical features in the set of medical features to differentiate patients in the advanced cohort from patients in the non-advanced cohort at various time-points based on the respective values of the medical features in an iterative manner.

12. The method of any of claim 1, wherein the predictive model identifies a particular feature as a predictive feature by:
determining, at each time-point in the multiple time-points, a first number of patients in the non-advanced cohort that have the particular feature, and a second number of patients in the advanced cohort that have the particular feature;
calculating, for each time-point, a respective delta value between the first number and the second number at the time-point; and
determining that a first delta value calculated for a first time-point is more than a second delta value calculated for a second time-point for more than a specific threshold value, and in response,
identifying the particular feature as a predictive feature.

13. A system comprising:
one or more computers and one or more storage devices storing instructions that are operable, when executed by the one or more computers, to cause the one or more computers to perform operations comprising:
receiving, by a system of one or more computers, a set of medical features, each feature in the set of medical features is measured at multiple points in time throughout a disease progression journey in a respective patient in a set of patients;
mapping each point in time to a respective time-point that represents the relativeness of the point in time to the overall disease progression journey of the patient;
forming, from the set of patients, an advanced cohort and a non-advanced cohort, the advanced cohort including patients whose disease progressed to a predetermined advanced form, the non-advanced cohort including patients whose disease did not progress to the predetermined advanced form;
applying a predictive model on the set of medical features to identify one or more predictive features that differentiate between the advanced cohort and the non-advanced cohort at various time-points; and
storing, in a data storage device, information of the one or more predictive features.

14. (canceled)

15. The system of claim 13, wherein the operations further comprise:
receiving, from a client device, data on medical features of a particular patient;
retrieving the information of the one or more predictive features from the storage device;
applying the predictive model on the data to predict a likelihood of the particular patient progressing to the advanced form of the disease based on the information of the one or more predictive features; and
transmitting the likelihood to the client device for presentation.

16. The system of claim 15, wherein the operations further comprise:
applying the predictive model on the data to predict a timeline for a progress of the disease in the particular patient; and
transmitting the timeline to the client device for presentation.

17. The system of claim 13, wherein the operations further comprise suggesting a medication or a medical procedure for the patient based on the likelihood.

18. A computer storage medium encoded with a computer program, the program comprising instructions that are operable, when executed by a data processing apparatus, to cause the data processing apparatus to perform operations comprising:
receiving, by a system of one or more computers, a set of medical features, each feature in the set of medical features is measured at multiple points in time throughout a disease progression journey in a respective patient in a set of patients;
mapping each point in time to a respective time-point that represents the relativeness of the point in time to the overall disease progression journey of the patient;
forming, from the set of patients, an advanced cohort and a non-advanced cohort, the advanced cohort including patients whose disease progressed to a predetermined advanced form, the non-advanced cohort including patients whose disease did not progress to the predetermined advanced form;
applying a predictive model on the set of medical features to identify one or more predictive features that differentiate between the advanced cohort and the non-advanced cohort at various time-points; and
storing, in a data storage device, information of the one or more predictive features.

19. The computer storage medium of claim 18, wherein the operations further comprise:
receiving, from a client device, data on medical features of a particular patient;
retrieving the information of the one or more predictive features from the storage device;
applying the predictive model on the data to predict a likelihood of the particular patient progressing to the advanced form of the disease based on the information of the one or more predictive features; and
transmitting the likelihood to the client device for presentation.

20. The computer storage medium of claim 19, wherein the operations further comprise:
applying the predictive model on the data to predict a timeline for a progress of the disease in the particular patient; and
transmitting the timeline to the client device for presentation.

21. The computer storage medium of claim 18, wherein the operations further comprise suggesting a medication or a medical procedure for the patient based on the likelihood.
